(19) United States
(12) Patent Application Publication
(10) Pub. No.: US 2019/0032068 A1
(43) Pub. Date: Jan. 31, 2019

(54) CONSTRUCT OF NUCLEIC ACIDS COMPRISING A PROMOTER LINKED TO A NUCLEOTIDE SEQUENCE ENCODING THE PRECURSOR OF miRNA 319e OF V. VINÍFERA; METHOD FOR REGULATING THE EXPRESSION OF A TARGET SEQUENCE IN A TARGET CELL AND ITS USE IN POST-TRANSCRIPTIONAL GENE SILENCING OF A TARGET SEQUENCE

(71) Applicants: INSTITUTO DE INVESTIGACIONES AGROPECUARIAS, Providencia (CL); BIOTECNOLOGÍA FRUTÍCOLA S.A., Vitacura (CL)

(72) Inventors: Humberto Godofredo Prieto Encalada, Vitacura (CL); Alvaro Gonzalo Castro Oyarzún, Maipú (CL)

(73) Assignees: INSTITUTO DE INVESTIGACIONES AGROPECUARIAS, Providencia (CL); BIOTECNOLOGÍA FRUTÍCOLA S.A., Vitacura (CL)

(21) Appl. No.: 16/029,287

(22) Filed: Jul. 6, 2018

(30) Foreign Application Priority Data
Jul. 7, 2017 (CL) CL 201701793

(51) Int. Cl. C12N 15/82; C12N 15/70; A01H 6/88

(52) U.S. Cl. CPC C12N 15/8237; C12N 15/8222; A01H 6/88; C12N 15/70

(57) ABSTRACT

A nucleic acid construct comprising a promoter operably linked to a nucleotide sequence coding for the precursor of Vitis vinifera miRNA 319e capable of generating an amiRNA hairpin structure that includes a complementary hypervariable sequence between 60-100% to a target sequence. A method of regulating the expression of a target sequence in a target cell comprising introducing the nucleic acid construct into a target cell, and using the construct in the post-transcriptional gene silencing of a target sequence in a constitutive and stable manner.

### DESCRIPTION OF THE INVENTION

The present invention refers to constructs of the artificial miRNA (miRNA) type (amiRNA) derived from miRNA 319e of Vitis vinifera (wi-miRNA319e) and its use as an agent for the silencing of genes by a post-transcriptional mechanism. In particular, the present invention refers to the use of the precursor RNA molecule and the active molecule corresponding to vitis-miRNA319e of Vitis vinifera, as a general tool for modulating the expression of genes through the generation of amiRNAs.

The present invention also refers to a method for regulating the expression of a target sequence in a cell, wherein this method comprises a) introducing a polynucleotide construct encoding the amiRNA precursor sequence capable of forming a double-stranded RNA or hairpin complementary to the sequence to be regulated: amiRNAs derived from the miRNA 319e of Vitis vinifera (vvi-miRNA319e), where this polynucleotide is bound to a transcription promoter by means of which the transcription of the antisense strand of amiRNA precursor is regulated according to claim 1 and (b) growing the target cell to which the construct was introduced according to (a) under the suitable conditions for the promoter to activate transcription of the antisense strand of the amiRNA precursor, produce the amiRNA and bind it to the target RNA to cleave it.

Additionally, the use of the nucleic acid construct comprising a promoter operably bound to a nucleotide sequence coding for the precursor of the artificial miRNA, pre-amiR319e, according to claim 1 is proposed, CHARACTERIZED for being useful for the post-transcriptional gene silencing of a target sequence.

The present invention is related to the field of molecular biology and biotechnology applied to the regulation of gene expression in plants. In particular, the present invention refers to nucleic acid constructs for the production of artificial microRNAs (amiRNA) from amiRNAs precursors.

RNA Interference as Tools of Post-Transcriptional Gene Silencing.

The mechanisms of gene silencing in eukaryotic cells and plants have been extensively studied over the last decade. From the information derived from the sequencing of different genomes and the emergence of tools for the study of molecular phenomena within the cell, it has been possible to characterize and understand the role of certain RNA molecules involved in the process of post-transcriptional regulation of gene expression.

The system by which RNAs suppress the expression of specific genes based on specific sequences is known as RNA interference (iRNA). There are different types of RNA interference molecules that are generated basically by the fragmentation of RNA precursors, among these are the siRNA (by its name in English small interfering RNA), the miRNA (by micro-RNA) and piRNA (piwi-interacting RNA).

The microRNAs (miRNA) were first described from a study in the Caenorhabditis elegans worm, more specifically, in a mutant species of this worm. After this study it was concluded that miRNAs corresponding to 21-nt sRNAs (nucleotides) that target messenger RNA molecules with complementary sequences can inhibit the translation of certain genes. Several criteria have been established to define whether a sRNA in a miRNA; first the sRNA sequences must be encoded in the respective genome, they must be transcribed from large precursor molecules and the precursor molecule must be processed to form a mature molecule. In addition, these miRNAs must have a target sequence named target mRNA, where a base pairing exists to specifically repress the translation of that gene or the cleavage of the mRNA.

The synthesis of miRNAs in plant cells has been mainly studied in Arabidopsis thaliana. The synthesis of miRNAs in plant cells begins from the processing of long precursors of pre-miRNAs (Bartel (2004 Cell 116: 281 297) by the enzyme DCL1, which cuts the precursor near the bottom to form an extensive stem-loop structure, releasing a RNA hairpin, which is cut again by the enzyme to generate a structure of approximately 21 nt (Kurihara and Watanabe, 2004). It is believed that this processing occurs in the nucleus of the cell, so that other proteins must transport the miRNAs to the cytoplasm where they form the gene silencing complexes by means of their binding to the RISC complex. In the RISC complex, it is the Argnoauta protein that acts as the main component, guiding the binding and recognition of the miRNA to its target mRNA.

Use of Artificial miRNAs to Suppress Gene Expression.

Due to the structural characteristics and the ability to inhibit specific target genes, miRNAs have been used as molecular tools for the post-translational inhibition of genes. From this, artificial miRNAs (amiRNA) have been designed and constructed according to the characteristics of the target gene to be silenced and of the target species.

For example, the use of gene silencing techniques based on amiRNAs has been previously described in Arabidopsis thaliana, Oryza sativa, Medicago truncatula and Chlamydomonas reinhardtii (Schwab et al., 2006; Warthmann et al., 2008; Alvarez et al., 2006). Molnar et al., 2009; Devers et al., 2013).

The design and construction of miRNA molecules for the modification of phenotypic characteristics in different plant species from the inclusion of artificial miRNAs in grafts have been described in various patent documents.

The document WO 2012140230 A1, for example, discloses a method for modifying the phenotype of a grafted plant, which comprises providing a rhizome, wherein the expression of a target gene is altered through transcriptional regulation mediated by an RNA in a non-coding region of this gene, and grafting this rhizome to generate a grafted plant, where this graft causes the transmission of transcriptional gene regulation mediated by RNA from the rhizome to the plant, thereby modifying the phenotype of the grafted plant. The type of construct proposed in this document corresponds to an RNA molecule comprising 19-25 nucleotides long.

Other documents have disclosed sequences of amiRNA and its precursors to inhibit the expression of different mRNAs in order to improve the productive capacity of crops (CN102220334 A), increase the tolerance of plants to environmental conditions such as salt tolerance (CN102220333 A and CN102220332 A), resistance to temperature (CN102220329 A), increase the growth of plants (WO2006034368 A2), in different crops such as rice and soybeans.

Methods of preparing amiRNAs from the design and synthesis of gene constructs encoding a recombinant miRNA, capable of reducing the level of mRNA of the target sequence in the plant of interest, have also been described. For example, the document WO 2013063487 A1 discloses a polynucleotide construct comprising: a) a first element comprising a recombinant expression construct containing a polynucleotide of interest having at least 80% identity with the target sequence and b) a second element comprising a recombinant miRNA construct, wherein this recombinant miRNA expression construct encodes a miRNA of 21 nucleotides (21-nt), wherein this miRNA, when expressed in the plant, is capable of reducing mRNA level of the target sequence.

In document WO2006073727 (A2) there is presented a method for providing a 22-nucleotide RNA molecule to a plant cell, which comprises the transcription of a DNA sequence encoding a miRNA precursor. This DNA encoding a miRNA precursor comprises a nucleotide sequence derived from the sequence of the miR167 precursor.

There are no antecedents related to the use of pre-miRNAs of V. vinifera up to now, the closest being the use of A. thaliana ath-miR319a and O. sativa osa-miR528 (Schwab et al., 2006; Al., 2010) for the design of artificial molecules of miRNAs.

Although the production of miRNAs derived from plants has been described, it is desirable to provide improved miRNA precursors, which allow to direct the silencing of a target gene by means of its constitutive expression, in order to generate stable transgenic lines.

miRNAs in the Silencing of Genes in Vitis Vinifera: Pre-miRNA of the miR319 Family.

Vitis vinifera, commonly known as grape, is one of the most cultivated vegetable species worldwide due to its industrial importance as a raw material for the production of wines and derivatives. Because of this, Vitis vinifera has also been studied for the detection of the presence of miRNAs and their manipulation to obtain improved strains in terms of production and fruit quality.

Recently, it has been described that there are more than 285 miRNAs in Vitis vinifera (Belli Kullan et al, 2015.). Of the miRNAs described, miRNAs belonging to the miR319 family: VVI-miR319 b, c, e, f and g stand out (Kozomara and Griffiths-Jones, 2014, Mica et al., 2009). On the other hand, it has been indicated that the mature forms of these miRNAs would be involved in the development and production of the grape (Belli Kullan et al., 2015). The simplest member in terms of size and structure belonging to the miR319 family, is the pre-miRNA VVI-miRNA319e (107 bases, http://www.mirbase.org/cgi-bin/mirna_summary.pl?org=vvi), which would present a relatively short and simple stem and loop structure, outlining this miRNA as a possible template for the design and construction of artificial miRNAs to be used in the post-transcriptional genetic modification of genes of interest.

Studies of gene silencing by miRNAs have shown that there are certain main pre-miRNA precursors, which undergo processing to generate the stem-loop structure characteristic of miRNAs. Among these are the precursors of the miR319 family, which have been used frequently in A. thaliana through the construction of an expression system of specific amiRNAs against the target gene to be silenced (Schwab et al., 2006).

For the construction of amiRNAs, the use of directed mutagenesis on the endogenous miRNA precursors has been proposed, by PCR overlapping, where the selected primers are able to replace (Schwab et al., 2006). The problem of this procedure is that it is required to perform more than one PCR reaction, at least 4 reactions, which involves a lot of time, high cost of reagents and the increase in the probabilities of errors in the process. Therefore, there is a need to generate new tools and approaches to design and build amiRNAs in a fast, efficient and simple way than the techniques proposed so far, applying it in a particular way to the design of pre-amiRNA precursors of interest such as miRNAs of the miR319 family for Vitis vinifera.

### DETAILED DESCRIPTION OF THE INVENTION

The present invention refers to constructs of the artificial miRNA type (amiRNA) derived from vitis-MIR319e from Vitis vinifera and its use as an agent for the silencing of genes by a post-transcriptional mechanism. The inventors have succeeded in designing a construct that allows generating the precursor of Vitis vinifera miRNA 319e (vvi-miR319e) as the basis for the production of miRNAs with hairpin RNA structure capable of binding to a target sequence and silencing its expression.

The use of wi-miR319e as a template allows faster and simpler production of miRNAs capable of silencing a target gene, due to its smaller size (107 bases), compared to the miRNA previously described, such as ath-miR319a (186 bases). In practice, this allows the generation of miRNAs without the use of additional templates, and the artificial miRNA319e precursor construct can be directly included in grafts on plants. Secondly, the inventors have designed a construct coding for the precursor miRNA319e in such a way that it is constitutively expressed in the target plant, allowing to generate stable transgenic lines.

The present invention is illustrated in its scope with 5 amiRNA constructs derived from the main construct vvi-MIR319e from Vitis vinifera (FIG. 2). The proposed pre-miRNAs were designed from the template of Vitis vinifera vvi-MIR319e, which was chosen from the miRBase database.

After amplifying and cloning the sequence of vvi-MIR319e from the DNA of the genome of ‘Thompson Seedless’ by PCR, the template sequence corresponding to a fragment of 214 bases was obtained whose sequence includes 107 bases corresponding to the pre-vvi-MIR319e between the bases 114 and 134 which will form the stem-loop structure.

Using the microRNA design tool WMD3-Web MicroRNA (Griffiths-Jones et al., 2008), mature miRNAs capable of recognizing areas in a reference gene, which in this case codes for two versions of green fluorescent protein, (GFP, mGFP and sGFP), which contain at least one nucleotide difference between both sequences in regions designated region A and region B of the gene, were selected. From this, five mature miRNAs were chosen and used for the specific design of modified vvi-MIR319e directed against the sGFP and mGFP isoforms, as an example of the target gene to be silenced.

The present invention also includes a nucleic acid construct comprising a promoter operably linked to a nucleotide sequence coding for the artificial miRNA precursor (amiRNA) pre-amiR319e which is folded to generate a hairpin RNA comprising a complementary hypervariable sequence and homologous by 60-100% with the target sequence. The operably linked promoter mediates the transcription of the antisense sequence of the amiRNA precursor.

When the present invention refers to the expression “target sequence, aim sequence or objective sequence”, they may be used interchangeably with reference to the nucleic acid sequence selected to suppress and/or repress its expression, without being limited to coding polynucleotide sequences of peptides and/or polypeptides. The target sequence includes a sequence that is substantially or completely complementary to the amiRNA.

The amiRNA hybridizes with the target sequence being between 60 and 100% complementary and homologous with it, being preferably between 60 and 80%, preferably 90% and more preferably 100% identical to the sequence. The complementary sequence corresponds to a hypervariable region, understood by this as a region that shows a constant pattern of variation of nucleic acids.

It is to be considered that by nucleic acid constructs assembled in an expression system part of the present invention it is possible to generate an artificial miRNA precursor (amiRNA) pre-amiR319e, which in turn will allow to produce a hairpin RNA structure comprising a hypervariable sequence complementary between 60-100% to the target sequence to be silenced. The hypervariable sequence part of the pre-amiR319e is located between nucleotides 10 to 31 and 76 to 96 of SEQ ID 1.

This hypervariable sequence allows the amiRNA to be contained with the sequence to silence any gene of any genus and species. Preferentially, the gene to be silenced corresponds to a gene of a plant cell, referring to “plant cell” in its broadest sense. In the present invention, the plant cell corresponds to plant cells of any species and type of plant, without being limited, to plant cells of woody, ornamental or decorative species, crops or cereals, fruits or vegetables and algae. Examples of plant cells correspond, without being limited, to cells derived from plants of the genus Vitis, Arabidopsis, Nicotiana, Oriza, Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nuphar, Pennisetum, Persea, Phaseolus, Physcomitrella, Picea, Pinus, Poncirus, Populus, Prunus, Robinia, Rose, Saccharum, Schedonorus, Secale, Sesamum, Solanum, Sorghum, Stevia, Thellungiella, Theobroma, Triphysaria, Triticum, Vitis, Zea, or Zinnia. Additionally, plant cells in the present invention may be any plant cell susceptible to transformation, including gymnosperm and angiosperm cells, both in monocotyledonous plants and in dicotyledonous plants. Between monocotyledonous angiosperms include but are not limited to barley, wheat, rice, onion, oats, rye and other cereals. In the case of dicotyledonous angiosperm, only examples are tomato, tobacco, cotton, beans, soybeans, peppers, lettuce, peas, alfalfa, clover, radish cabbage, carrot, beet, eggplant, spinach, cucumber, squash, melon, sunflower and various ornamental plants. Examples of woody species include poplar, pine, redwood, cedar, oak, among others.

The term “plant-derived plant cell” may correspond to a cell of an entire plant, a part of the plant, or part of organs (e.g., leaves, stems, roots, etc.), a plant cell, or a group of plant cells, such as plant tissues, plant seeds and their progeny. Seedlings are also included in the sense of “plant”.

The pre-amiRNA of the target sequence can be included, in a preferential form of the invention, in the expression vector pGWB502 (Nakagawa et al., 2007). The expression vector can be included in a compatible bacterial cell for its amplification and for its transfer to plants.

In the present application, when it refers to “artificial miRNA” or “amiRNA” it refers to a small ribonucleic acid of a length of between 19-25 nucleotides, corresponding to an artificial DNA, that is, it does not occur naturally, and that functionally it is capable of inhibiting the transcription of a target RNA to be silenced.

An amiRNA is produced from an “amiRNA precursor” which corresponds to a polynucleotide of greater length than that of a native pre-miRNA, and which, after its cellular processing, is able to generate a mature amiRNA. In particular, the amiRNA precursor corresponds to a modified miRNA. In the present application, the precursor of an amiRNA is named pre-amiRNA.

In the present application, when referring to “nucleic acid construct” or “construct”, it refers to a particular polynucleotide which comprises a combination of sequences that can be transcribed to generate a double-stranded RNA or a hairpin structure. This construct can be introduced into a host cell in such a way as to produce the predicted amiRNA. In particular, the construct comprises a promoter, a sequence that mediates the regulation of expression and facilitates the modulation of the gene, and the genes associated with the expression of the amiRNA precursor. When referring to “host cells” corresponds to a cell having the characteristics necessary for a nucleic acid construct to be introduced, being able to express and replicate the construct. The host cells can be a prokaryotic cell, preferentially Escherichia coli, or a eukaryotic cell.

Generally, the term “inhibition or silencing” refers to a decrease or reduction in the expression of a target sequence or objective, which may be total or partial.

When referring to “operably linked”, it refers to a sequence between a promoter and a second sequence which is functionally linked. Regarding the promoter, it corresponds to a DNA sequence to be recognized by the RNA polymerase where transcription starts. The promoter mediates or initiates the transcription of a DNA sequence to a RNA.

On the other hand, when reference is made to a vector, it corresponds to a nucleic acid used to introduce the nucleic acid construct or polynucleotides to the host cell. In particular, an expression vector corresponds to a vector that contains the sequences and components necessary to allow the transcription of the nucleic acid introduced into the host cell.

The present invention also refers to a method for regulating the expression of an target sequence in a cell, wherein this method comprises a) introducing a polynucleotide construct encoding the precursor of amiRNA sequences capable of forming a double-stranded RNA or hairpin complementary to the regular sequence, where the polynucleotide is bound to a transcription promoter by means of which the transcription of the antisense strand of the amiRNA precursor is regulated and (b) growing the cell (a) under the appropriate conditions for the promoter to activate the transcription of the antisense strand of the amiRNA precursor, the amiRNA is produced, binds to the RNA and cleaves it.

The present invention further relates to the use of the nucleic acid construct comprising a promoter operably linked to a nucleotide sequence encoding the precursor of the artificial miRNA (amiRNA) pre-amiR319e, because it is useful in the post-transcriptional gene silencing of a target sequence.

Additionally, the use of the artificial miRNA319e precursor construct for post transcriptional gene silencing through its insertion into the plant in areas of grafting is proposed. The artificial miRNA319e precursor construct can be included in grafts on plants to constitutively express and generate stable transgenic lines. The silencing signals correspond to mobile signals, capable of modifying the phenotype of the rootstock. The present invention also includes in its scope the use of the artificial miRNA319e precursor construct for post-transcriptional gene silencing including in plants to be constitutively expressed and generate stable transgenic lines. The proposed use includes the incorporation of the construct by means of grafts on plants, through its sprinkling on the target plant, grafting, and/or directly on the “franca” plant without grafting. It can also be applied through various preparations containing the amiRNA already generated for application in diets.

The application examples presented below illustrate a form of the present invention, without limiting its scope to other plant cells, other than Vitis vinifera.

### DESCRIPTION OF FIGURES

FIG. 1.—Vitis vinifera miR319e from “Thompson seedless”. A) Pre-miR319e molecule sequence. B) miRNA hairpin model expected for pre-miR319e sequence of ‘Thompson Seedless’.

FIG. 2.—Artificial vvi-pre-miR319e molecules model with target sequence against GFP gene designed by the microRNAs design tool WMD3-Web MicroRNA.

FIG. 3.—Two-step PCR for the design of artificial vvi-pre-miR319e against GFP gene (amiR319e-GFP). A) Design strategy of artificial vvi-pre-miR319e against GFP gene by two-step PCR using “Overlapping long-primers”presented therein. B) amiR319e-GFP structures, where the dark blue areas represent the 21-nucleotide molecule amiR319e-GFP, the light blue areas represent the amiR*319e molecule of 21 nucleotides and the underlined nucleotides represent the non-modifiable residue bases.

FIG. 4.—Functional evaluation of amiR-319e-GFP in transgenic plants of Nicotiana benthamiana. A) Functional analysis of the amiR319e-GFP gene in N. benthamiana plants double transformed from genomic DNA samples (left) and cDNA (right) using primers for the kanamycin resistance gene (nptII gene in GFP cassette transformation), Hygromycin resistance gene (hptII gene present in cassete transformation amiR319e-GFP) and green fluorescent protein gene (gfp gene present in GFP cassete transformation). B) Analysis of fluorescence emission of GFP in transformed plants and control lines without intervention (wild type).

FIG. 5.—Grafting test of plants expressing MIR B mGFP on mGFP plants. A) Grafting of Vitis vinifera ‘Thompson Seedless’ plants constitutively expressing MIR B mGFP (digitally red colored) on plants constitutively expressing mGFP. B) Analysis of GFP expression at 30 days post-graft by epifluorescence microscopy. Patterns 1 and 2 correspond to leaves located under the graft in the region immediately adjacent to this, pattern 3 corresponds to a leaf located in a region far from the graft area. C) Quantification of the red (chlorophyll emission) and green (green fluorescent protein emission) channels of the microphotographs.

FIG. 6.—Evaluation of mGFP expression after removing MIR B mGFP graft to evaluate the persistence and/or stability of the graft induced silencing. A) Grafted plant (mGFP pattern+MIR B mGFP graft) in a biosecurity greenhouse allowing it to enter in recess, (B) Verification of the grafted region regarding the new generation of sprouts. C) Evaluation of GFP expression in the leaves formed during the spring in the area corresponding to the pattern after 8 months post-graft. (D) Quantification of the red (chlorophyll emission) and green (green fluorescent protein emission) channels of the microphotographs.

### EXAMPLES

### Example 1

### Design of Artificial vvi miR319e

Cloning of miR319e Gene and Target Gene Selection

The design and construction of the pre-miRNAs was performed from the pre-miRNA template of Vitis vinifera, vvi-MIR319e chosen from the miRBase database. The sequence of vvi-MIR319e was cloned from the genome DNA of ‘Thompson Seedless’ by PCR and the sequence data from the V. vinifera PN400 genome reference (Jaillon et al., 2007). The segment of the genome containing the pre-miR319e sequence was amplified by PCR using the following primers:

TABLE 1
Pre-miR319e primers

| Primer | Sequence | Length |
|---|---|---|
| 319e-forward | 5′ CACAACTTTCACTATGGATG 3′ | 20 |
| 319e-reverse | 5′ GGAAAAGAGAAGAACTAGGAG 3′ | 21 |

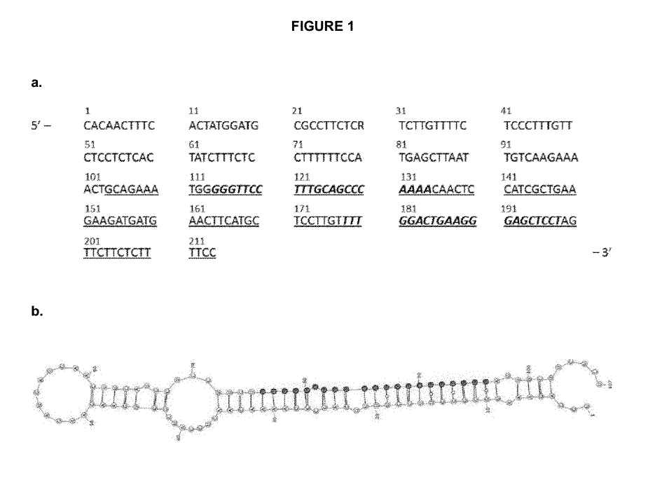

PCR amplification was performed according to the following conditions: 94° C. for 2 min; 35 cycles of 94° C. for 30 s, 55° C. for 30 s, and 72° C. for 30 s; and a final elongation at a temperature of 72° C. for 2 min. The obtained PCR product corresponds to a fragment of 214 bases (FIG. 1a), whose sequence includes 107 bases corresponding to the pre-vvi-MIR319e between bases 114 and 134 (sequence underlined in FIG. 1a). This sequence will form the stem-loop structure predicted in FIG. 1b).

The obtained PCR product was cloned into the pGEMT Easy vector (Promega Corporation, Madison, Wis., USA) and sequenced for later use.

To evaluate the phenomenon of post-transcriptional silencing, a modified version of the GFP mGFP5-ER gene will be considered as a target sequence (Siemering et al., 1996, NCBI Acc. Number U87974). By means of the microRNA design tool WMD3-Web MicroRNA (Griffiths-Jones et al., 2008), mature miRNAs capable of recognizing areas containing at least one nucleotide difference between both sequences at position 10 (region A) or 11 (region B) were selected. In this way, two favorable regions were used to design the modified vvi-MIR 319e.

Five mature miRNAs used for the specific design of modified vvi-MIR319e directed against the sGFP and mGFP isoforms were chosen. These include the mature miRNAs designed to recognize the A region: MIR A sGFP: corresponds to the complementary sequence of the sGFP isoform, MIR A mGFP: corresponds to the complementary sequence of the mGFP isoform, MIR A.1 mGFP: corresponds to the sequence that has almost perfect complementarity with the sGFP isoform and only the nucleotide at position 10 is complementary to the mGFP isoform and the mature miRNAs designed to recognize the B region (between nucleotides 743 and 763 of the alignment). For region B, two mature miRNAs were used: MIR B mGFP with perfect complementarity with mGFP and MIR B sGFP, which presents perfect complementarity with the sGFP isoform.

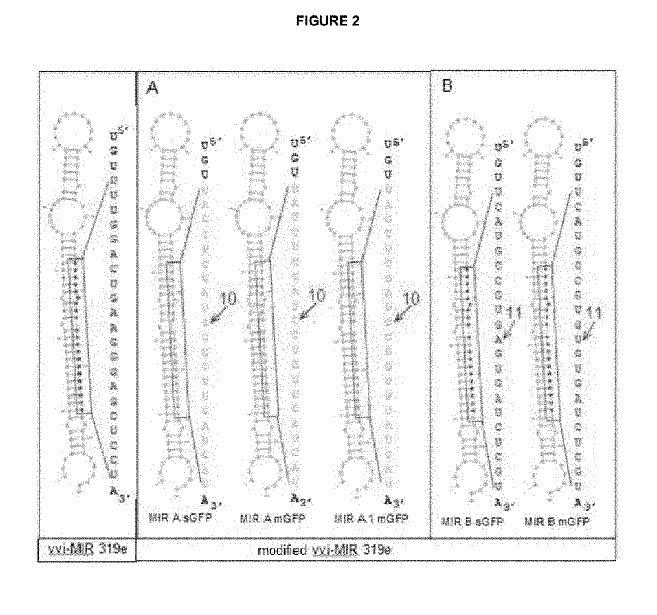

After the design and construction of the pre-miRNA, its secondary structure was analyzed by the mfold folding analysis tool (Zuker, 2003), available at http://mfold.rna.albany.edu/?q=mfold, obtaining 5 possible structures (FIG. 2).

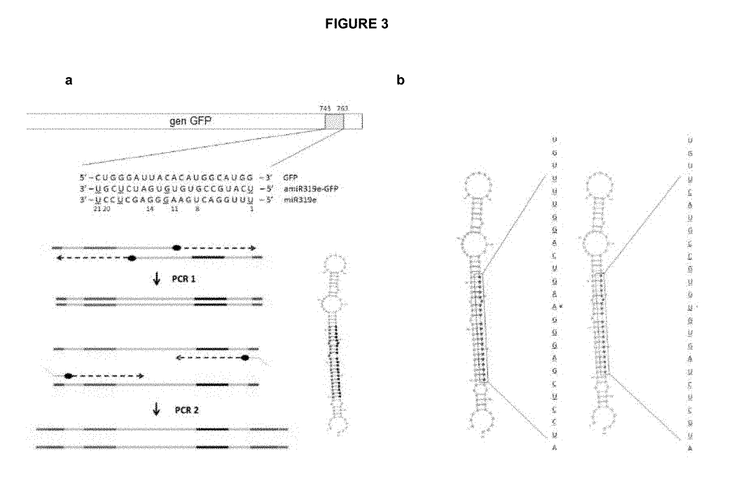

To continue the tests, a mature 21-nt miRNA was selected, which includes a sequence for detecting the GFP coding sequences between residues 743 and 763 (FIG. 3a).

From this, the primers of the long type primer were designed. A 2-step PCR was performed (FIG. 3a) to produce the pre-miRNA sequences that make it possible to obtain an amiR319e with structural and spatial characteristics (folding) that make it possible to form the characteristic stem-loop structure (FIG. 3b).

### Example 2

### Synthesis of Artificial vvi miR319e and Assembly in Expression System

Synthesis of Artificial Pre-amiR319e V. Vinifera.

A pre-amiR319e was constructed using two-step PCR by means of partial overlapping of 2 primers of the long type primers:

| Primer | Sequence | Length |
|---|---|---|
| Long- | 5′ AAAAAGCAGGCTGAGCTCTGCA | 87 |
| primer | GAAATGCGGGATCATACGATGTCAT | |
| 1 | GAACAACTCCATCGCTGAAGAAGAT | |
| | GATGAACTTCATGCT 3′ | |
| Long- | 5′ AGAAAGCTGGGTGAGCTCAGAG | 81 |
| primer | AAGAACTACGAGATCACACATGGCA | |
| 2 | TGAACAAGGAGCATGAAGTTCATCA | |
| | TCTTCTTCA 3′ | |

Structurally, three parts of the pre-amiR319e must be considered for its synthesis: a) the miRNA region, b) the miR319e framework, and c) Borders and boundaries. The considerations for the design and construction of each component of the pre-amiR319e are detailed below.

a) miRNA Region

This corresponds to a 21-nt miRNA that includes the pre-amiR319e sequence for GFP. For the construction of this component, the following requirements were considered: a) the conservation of the unpaired bases, associated with the conformation of the main stem of the pre-miR319e, and b) the conservation of the G-U bonds that form the main stem.

Once the GFP-miRNA of 21-nt was selected, it was modified in positions 1, 12, 18, and 21 (5′-3 ′) by U, G, U, and U, respectively. These bases should be considered as non-modifiable for all miRNAs based on miR319e.

b) Backbone Sequence

For the constitution of the construct backbone, sequences 1-9, 32-75 and 97-107 were maintained.

c) Borders or Boundaries Sequences

The 5′ and 3′ flanking regions defined by starters long-primers included two additional sequences: Sac I restriction sites and attB recombination signals at the 5′ and 3′ ends. The starters long-primers were designed by the automated web tool for the design of starters for VVI-miR319e pre-miRNA. This tool is available at:

http://www.flujogenico.cl/plantbiotech/amir319edesigner.

Starters Long-Primers Sequences:

| | |
|---|---|
| Long- | 5′ AAAAAGCAGGCTGAGCTCTGCAGAAATGCGGGA |
| primer 1 | TCATACGATGTCATGAACAACTECCATCGCTGAAGA |
| | AGATGATGAACTTCATGCT 3′ |
| Long- | 5′ AGAAAGCTGGGTGAGCTCAGAGAAGAACTACGA |
| primer 2 | TCACACATGGCATGAACAAGGAGCATGAAGTTCATC |
| | ATCTTCTTCA 3′ |

Pre-amiR319e Construction

The pre-amiR319e was constructed by means of a two-stage PCR:

Amplification step 1: A reaction mixture containing 0.5 U of KAPAHiFi (KAPA Biosystems, Wilmington, Mass., USA), 0.3 mM dNTPs (Promega Corporation, Madison, Wis., USA), 1× Fidelity Buffer with MgCl₂ and 50 pmol of each Long-primer, obtaining a final volume of 25 μL, was prepared. The PCR procedure considered the following program: 94° C. for 2 min; 10 cycles of 94° C. for 15 s, 55° C. for 30 s, and 72° C. for 15 s, and a final elongation at 72° C. for 30 s. An amplification product named stage 1 product was obtained.

Amplification stage 2: A second round of amplification was carried out from the product obtained in stage 1, this with the aim of completing the sequence of the attBs signal necessary for recombination in the vector pDONR207. This step consisted in the preparation of a reaction mixture containing 0.5 U of KAPA HiFi (KAPA Biosystems), 0.3 mM dNTPs (Promega Corporation), 1× Fidelity Buffer with MgCl2, 7.5 pmol of each primer: attB-F5′GGGGACAAGTTTGTACAAAAAAGCAGGCTTC 3′ and attB-R5′GGGGACCACTTTGTACAAGAAAGCTGGGT 3′, and 10 μL of product obtained in step 1. The PCR procedure considered the following program: 94° C. for 1 min; 5 cycles of 94° C. for 15 s, 45° C. for 30 s, and 72° C. for 20 s; 20 cycles of 94° C. for 15 s, 60° C. for 30 s, and 72° C. for 20 s; and a final extension at 72° C. for 1 min. The final amplification product was resolved on a 1.5% UltraPure agarose gel (Thermo Fisher Scientific) and visualized by staining with ethidium bromide. The band of interest was recovered by extraction from the gel using the Zymoclean Gel DNA kit (Zymo Research) according to the manufacturer's instructions. The product obtained is named stage 2 product.

Recombination of the Artificial Pre-miRNA in Vector pDONR207

The PCR product obtained from the second amplification was incorporated into the vector pDONR207 by the Clonase BP Gateway System (Thermo Fisher Scientific), according to the manufacturer's protocol. From this reaction results a vector containing the pre-miRNA-GFP (pDONR-pre-amiRNA-GFP), of which an aliquot was used to transform Escherichia coli One Shot TOP 10 competent cells (Thermo Fisher Scientific), following the manufacturer's instructions. From this, transformed cells were selected by incubation in LB medium supplemented with 15 mg/L of gentamicin overnight at 37° C. The selected clones were cultured in 5 mL of LB medium supplemented with 100 mg/L of Spectinomycin at 37° C. overnight with shaking at 180 rpm. Then, the cultures were centrifuged at 8000 g and DNA extraction was performed by means of the Zyppy plasmid Miniprep kit (Zymo Research). Plasmid DNA was checked by PCR and by restriction enzyme analysis. This PCR was carried out using the amiRNA F and amiRNA R primers (Table 1), in a PCR reaction that considered the following program: 94° C. for 2 min; 35 cycles of 94° C. for 15 s, 60° C. for 30 s, and 72° C. for 20 s; and a final extension at 72° C. for 1 min. Restriction analysis was carried out by incubation of 10 U of Sac I (New England Biolabs, USA), 1× NEB1.1 (New England BioLabs), 1 g/L of purified BSA and 500 ng of plasmid, leaving the reaction overnight at 37° C. The restriction assay was resolved on a 1.5% agarose gel and visualized by staining with ethidium bromide. The selected pDONR-pre-amiRNA-GFP plasmid was confirmed by sequencing (Macrogen).

Preparation of Pre-amiRNA-GFP Expression Vector

The pDONR-pre-amiRNA-GPF vector was recombined into the expression vector pGWB502 (Nakagawa et al., 2007). To achieve recombination, 150 ng of pDONR-pre-amiRNA-GFP and 150 ng of pGWB502 were mixed, using the Clonase LR Gateway System (Thermo Fisher Scientific) according to the manufacturer's instructions. The recombination mixture was used to transform E. coli One Shot TOP 10 competent cells, and positive clones were selected in LB medium supplemented with 100 mg/L spectinomycin. Positive clones were verified by PCR and restriction analysis using 10 U of Sac I enzyme (New England Biolabs, USA) and 1× NEB2. The resulting vector was named PGWB-pre-amiRNA-GFP.

### Example 3

### Transformation of Nicotiana Benthamiana with Rhizobium Radiobacter Comprising the Pre-amiRNAs Constructed

This example shows the procedure carried out for the preparation of Rhizobium radiobacter recombinant cells comprising the constructs coding for the pre-amiRNA and its use for transforming Nicotiana benthamiana and generating transgenic lines.

Preparation of Rhizobium Radiobacter (Example: Agrobacterium Tumefaciens)

Electrocompetent Rhizobium radiobacter GV3101 cells were prepared and electroporated with the constructs PGWB-pre-miRNA GFP and pB1121-mGFP5ER according to the protocol presented by McCormac et al. (1998). Electroporation was carried out in a Gene Pulser II system (Bio-Rad, Hercules, Calif., USA) adjusted to 1.25 kV, 25 μFD and 400Ω. The success of the transformation was determined by PCR of colonies and restriction analysis.

Recombinant Rhizobium cells containing the constructs PGWB-pre-amiRNA-GFP and pB1121-mGFP5ER were incubated overnight in LB medium supplemented with 100 mg/L of spectinomycin and 50 mg/L of kanamycin, respectively, at 28° C. and 180 rpm. An aliquot of this culture (50 μL) was transferred to 150 mL of LB medium supplemented with the corresponding antibiotic and incubated at 28° C. with shaking at 180 rpm until the culture reached an OD600 of 0.3. The cells were centrifuged for 10 min at room temperature and 3,000 g, to be resuspended in 40 mL of liquid MS medium (Murashigue and Skoog, 1962) supplemented with 0.5 μL of a 1M solution of acetosyringone (Sigma-Aldrich, St. Louis, Mich., USA), forming a suspension. The suspension was maintained at room temperature for 30 to 60 min before co-culture.

Preparation and Transformation of Nicotiana Benthamiana

A modified protocol was used to transform Nicotiana benthamiana from that described by Sun et al. (2006).

From this, Nicotiana seeds were disinfected by immersion in bleach (10% bleach plus 3 drops of Tween-20) for 10 min and washed 6 times with water for 1 min each wash. The disinfected seeds were hydrated for 2 h in water with occasional agitation. Seeds were dried using filter papers, seeded in Petri dishes containing MSO solid medium (MS medium (Murashige and Skoog, 1962) containing 15 g/L of sucrose and 3.2 g/L of phytagel, pH 5.8) and incubated for 7 days in the dark at 25° C.

Then, the seeds were subjected to incubation in a photoperiod of 16 h/8 h (light/dark) until the germination of these. After germination, the seedlings were co-incubated with the Rhizobium suspensions that include PGWB-pre-amiRNA GFP and pBI121-mGFP5ER.

The plants and bacterial suspensions were subjected to vacuum (20 mm Hg) for 1 min. After this, the cotyledon segments of the plants were dried with filter paper and seeded in Petri dishes with MS1 solid medium (MS salts medium, 30 g/L sucrose, and 1.5 mg/L zeatin, pH 5.8), being incubated for 48 h in darkness at 25° C.

Regeneration and Rooting

From the transformed plants the cotyledons were obtained and plated in Petri dishes with solid MS2 medium (MS medium containing 30 g/L of sucrose, 1.5 mg/L of zeatin, 250 mg/L of timentin, 200 mg/L of carbenicillin, 200 mg/L of cefotaxime, 50 mg/L of hygromycin, and 300 mg/L of kanamycin, pH 5.2). The cotyledons were transferred to fresh medium every 7 d until the time of callus formation. The calli were transferred to MS3 medium (MS medium containing 30 g/L of sucrose, 1 mg/L of zeatin, 250 mg/L of timentin, 200 mg/L of carbenicillin, 200 mg/L of cefotaxime, 50 mg/L of hygromycin B, and 300 mg/L of kanamycin) until the time of flowering (3-4 weeks). The shoots were transferred to MS4 medium (MS medium containing 30 g/L of sucrose, 250 mg/L of timentin, 200 mg/L of carbenicillin, 200 mg/L of cefotaxime, 25 mg/L of hygromycin B, and 150 mg/L of kanamycin), and were grown until the time of root formation (2-3 weeks).

After one month of culture, the specimens were transplanted into individual plastic bags of 350 mL with 150 mL of grass. The plants were covered with similar transparent plastic bags to prevent dehydration of the plant. After three weeks under these conditions, the plants were adapted for analysis.

### Example 4

### Analysis of the Transgene Conditions and Evaluation of the Silencing by the Pre-amiRNA Designed

From the protocol described in Example 3, three transgenic lines of N. benthamiana were generated: one for GFP, one for amiR319e-GFP and one comprising the GFP construct plus amiR319e-GFP. The three lines were initially characterized to confirm their transgenic status.

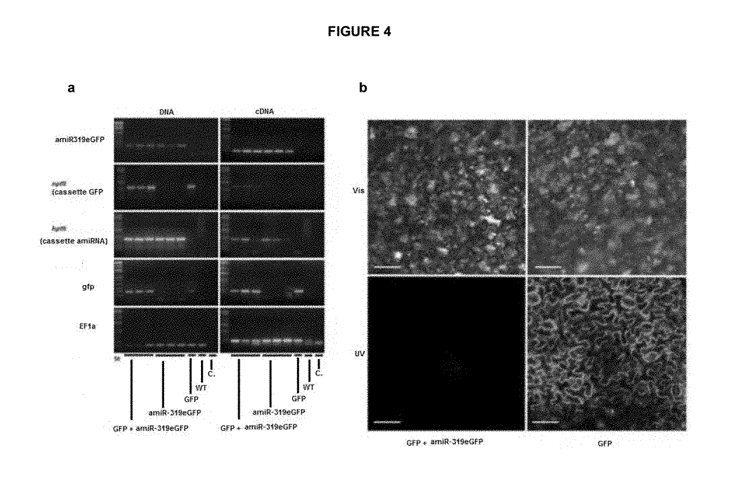

First, the genomic DNA of the transformed N. benthamiana plants was extracted for characterization by means of PCR. For this purpose, specific primers were used against the GFP gene: mGFP-F 5′ACAGATCTTCGATTTCAAGGAGGACGGAA 3′and mGFP-R 5′CCAGGCCTTCATGTTTGTATAGTTCATCCATGC 3, nptII gene (NPTII F 5′AGGCTATTCGGCTATGACTGG 3′and NPTII R′ 5′ATACCGTAAAGCACGAGGAAGC 3), gene MIR319 (PreamiRNA-F 5′GAGCTCTGCAGAAATGCGGGATCATA 3′ tyPre-amiRNA-R 5′GAGCTCAGAGAAGAACTACGAGATCA 3), hptII gene (HPT-F and HPT-R), and EF1α gene of N. benthamiana. The PCR reaction performed included 1× Green GoTaq Buffer, 1.25 mM MgCl2, 0.25 mM dNTPs, 0.6 U of GoTaq Flexi DNA Polymerase (Promega Corporation) and 10 pmol of each primer. The PCR program consisted of: 95° C. for 3 min; 30 cycles of 94° C. for 30 s, 59° C. for 30 s, and 72° C. for 30 s; and a final extension stage at 72° C. for 2 min. The analysis of the different genes by PCR of the three transgenic lines confirmed the transgenic status of the plants for each case, as appropriate (FIG. 4a).

In parallel, GFP emission was determined by fluorescence microscopy in an HCS LSI microscope (Leica Microsystems, Inc., Buffalo Grove, Ill., USA). From the analysis of GFP emission, the presence of GFP fluorescence was observed only in the plants transformed individually with GFP, the samples of plants transformed with GFP plus amiR319e-GFP lacking fluorescence. Control lines without intervention (wild type) do not present GFP emission and the lines transformed only with amiR319e-GFP either (FIG. 4b).

Analysis of Target Messenger RNA (mRNA)

A rapid detection analysis of GFP mRNA in the transgenic plants was carried out by the technique “modified 5′ rapid amplification of 5′ cDNA ends (5′ RACE)”. The mRNA content was extracted and purified by Dynabeads of oligo (dT) 25 (Thermo Fisher Scientific) according to the manufacturer's instructions.

Then, a DNA adapter was ligated to the mRNA by the mixture of the adapter sequence and the mRNA. The ligation reaction was performed by mixing 1× T4 RNA ligase buffer (Thermo Fisher Scientific), 1 mM ATP, 24 U of RNAs in Plus RNase inhibitor (Promega Corporation, USA) and 5 U of T4 RNA ligase (Thermo Fisher Scientific). This mixture was incubated for 1 h at 37° C., and the mRNAs bound to adapters were re-purified using Dynabeads. The isolated nucleic acids were resuspended in 10 μL of 10 mM Tris-HCl.

The mRNAs linked to adapters were used as templates to generate their respective cDNA. For this reaction, 10 μL of the mRNAs bound to adapters were added to a mix reaction containing 0.4 mM of each dNTP and 0.5 μg of an oligo (dT) 15 primer (Promega Corporation). The mixture was denatured for 5 min at 65° C. and placed on ice. Then, 1× SuperScript First-Strand Buffer (Thermo Fisher Scientific), 24 U of RNasin plus RNase inhibitor (Promega Corporation), 10 mM of DTT, and 200 U of Superscript II RT (Thermo Fisher Scientific) were added to the mixture denatured. The reaction was incubated for 10 min at 25° C., 1 h at 42° C. and 10 min at 70° C. Finally, 0.3 U of Ribonuclease H (Promega Corporation) was added and the mixture was incubated for 30 min at 37° C.

Following the protocol, the cDNA subjected to reverse transcription was amplified by PCR using the mGFP-F and -R primers (for the GFP gene), NPTII-F and -R (for the NPTII gene), and Pre-amiRNA-F and -R (pre-amiRNA319e-GFP). For PCR amplification, reaction mixtures were prepared with 1× KAPA Taq buffer plus Mg2₊ (KAPA Biosystems), 0.2 mM dNTPs (10 pmol of each primer), 0.4 U of KAPATaq polymerase (KAPA Biosystems) and 1 μL of a 1:5 dilution of cDNA. The PCR program included: denaturation at 95° C. for 3 min and 30 cycles of 30 s at 94° C., 30 s at 60° C. (for the N. benthamiana pre-amiRNA and NPTII genes) or at 57° C. (for the GFP gene), and 30 s at 72° C. A final extension was performed for 1 min at 72° C. The amplified products were resolved on 1.5% agarose gels and visualized by staining with ethidium bromide.

Analysis of amiRNA319e-GFP.

RT-PCR was used to detect the final loop structures of the pre-amiR319e-GFP miRNA in the plant. All procedures were modified from Varkonyi-Gasic et al. (2007), starting with 100 mg of fresh leaves. The process begins with the extraction of total RNA from the plant. The primers used in this case for RT-PCR were designed according to Chen et al. (2005). From the total RNA, 500 nanograms were taken and mixed with 1 μL of each stem-loop primer (Stock 10 mM) and 0.5 μL of each dNTP (10 mM). The mixture was incubated for 5 min at 65° C. and then cooled on ice for 2 min. After this, 4 μL of 5× First Strand buffer (Thermo Fischer Scientific) was added and mixed with 2 μL of 0.1 M DTT, 0.1 μL of RNase-OUT (40 U/μL) (Invitrogen, USA) and 0.25 μL of Superscript II reverse transcriptase (200 U/μL) (Thermo Fischer Scientific). The mixture was centrifuged, and it was included in an Eppendorf Mastercycler Nexus thermocycler (Thermo Fisher Scientific), undergoing the following program: 30 min at 16° C. and 60 cycles of 30 s at 30° C., 30 s at 42° C. and 1 s 50° C. Finally, the mix reaction was incubated for 5 min at 85° C. The amplified product was used as a template for a second PCR in which the universal RT-PCR primer was mixed with the previously described miR319e-GFP forward or miR166c forward primers. The PCR conditions were 3 minutes at 95° C. and 35 cycles of 30 s at 95° C., 30 seconds at 60° C. and 30 s at 72° C. A final extension was applied for 5 min at 72° C. The PCR products were separated by agarose gel electrophoresis using 3% agarose.

### Example 5

### Mobility Evaluation of Pre-amiR319e Silencing Signals

This example presents the mobility evaluation of silencing signals generated by MIR B mGFP. For this, a line of Vitis vinifera L. “Thompson Seedless” was developed that will express the miRNA constitutively.

In order to determine the capacity of movement and transmission of gene silencing, the line produced was used in grafting trials on stable transgenic vine plants for mGFP.

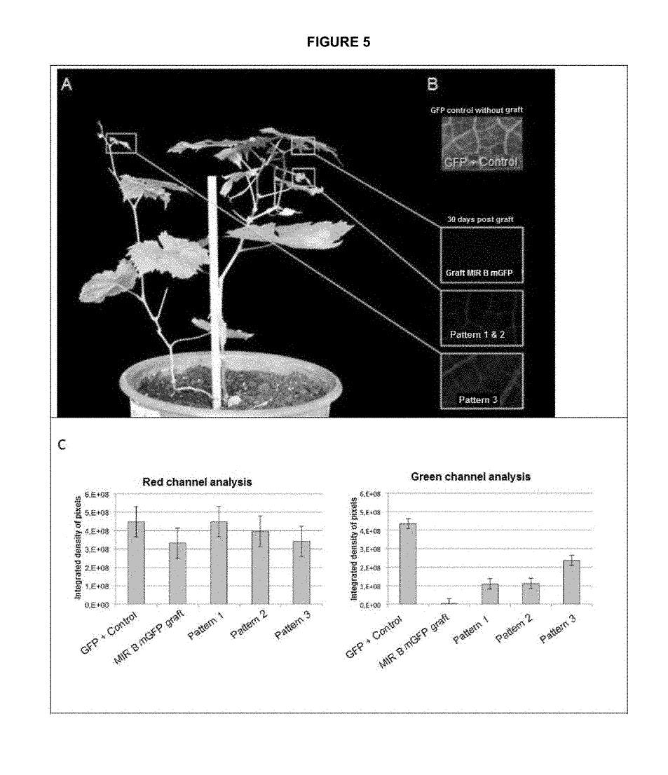

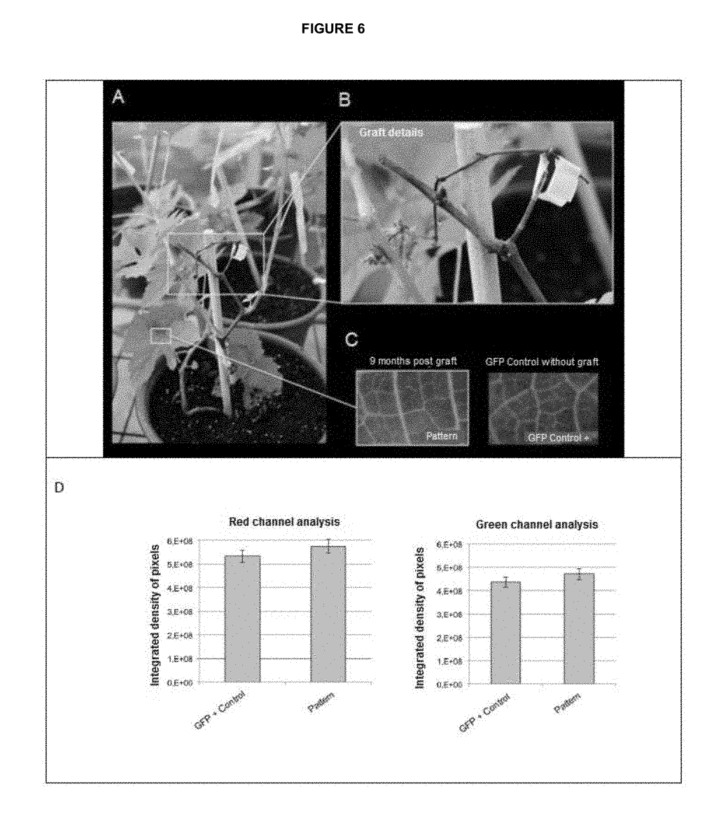

From the experiment, the results generated showed a phenomenon of bidirectionality of movement of the silencing signals, resulting more efficient the movement from graft to the rootstock. Changes in the phenotype were observed from green fluorescent protein expression under epifluorescence microscopy, when using as a graft (upper part) the line that generates the silencing signals and as a rootstock the line that expresses constitutively the mGFP isoform (FIG. 5). When the graft was removed, after 8 months post-graft, it was observed that the fluorescence in the rootstock (green channel emission) was similar to that of a control plant (mGFP) (FIG. 6).

Sequences
SEQ 1.
(SEQ ID NO 1)

| 5′- | 1 11 21 31 | |
|---|---|---|
| | CACAACTTTC ACTATGGATG CGCCTTCTCR TCTTGTTTTTC | |
| | 41 51 61 71 | |
| | TCCCTTTGTT CTCCTCTCAC TATCTTTCTC CTTTTTTCCA | |
| | 81 91 101 111 | |
| | TGAGCTTAAT TGTCAAGAAA ACTGCAGAAA TGGGGGTTCC | |
| | 121 131 141 151 | |
| | TTTGCAGCCC AAAACAACTC CATCGCTGAA GAAGATGATG | |
| | 161 171 181 191 | |
| | AACTTCATGC TCCTTGTTTT GGACTGAAGG GAGCTCCTAG | |
| | 201 211 | |
| | TTCTTCTCTT TTCC | -3′ |

1. A nucleic acid construct comprising a promoter operatively linked to a coding nucleotide sequence, wherein the coding sequence generates the miRNA 319e precursor of Vitis vinifera.

2. The nucleic acid construct comprising a promoter operatively linked to a coding nucleotide sequence according to claim 1, wherein the construct is capable of generating an amiRNA hairpin structure that includes a hypervariable sequence complementary between 60-100% to a target sequence.

3. The nucleic acid construct comprising a promoter operably linked to a coding nucleotide sequence according to claim 1, wherein the sequence of the amiRNA hairpin structure comprises a hypervariable sequence complementary between 60-100% to a target sequence is located between nucleotides 10 to 31 and 76 to 96 of SEQ ID 1.

4. A method for regulating the expression of a target sequence in a cell, said method comprising:
a) introducing a nucleic acid construct comprising a promoter operatively linked to a coding nucleotide sequence according to claim 1, wherein the sequence is linked to a transcription promoter so that the transcription of the antisense strand of the precursor is regulated; and
(b) growing the target cell to which the construct was introduced according to step (a) under the appropriate conditions for the promoter to activate the antisense strand transcription of the amiRNA precursor, the amiRNA is produced, and it binds to the target RNA to cleave it.

5. The nucleic acid construct comprising a promoter operably linked to a coding nucleotide sequence according to claim 1, wherein the construct is used in the post-transcriptional gene silencing of a target sequence.

6. The nucleic acid construct comprising a promoter operably linked to a coding nucleotide sequence according to claim 1, wherein the construct is included in plants to be constitutively expressed and generate stable transgenic lines.

7. The nucleic acid construct comprising a promoter operably linked to a coding nucleotide sequence according to claim 1, wherein the construct is included as grafts on plants.

8. The nucleic acid construct comprising a promoter operably linked to a coding nucleotide sequence according to claim 1, wherein the construct is included in plants by sprinkling the construct on the plants.

9. The nucleic acid construct comprising a promoter operably linked to a coding nucleotide sequence according to claim 1, wherein the construct is included in a “franca plant” without grafting.


SEQUENCE LISTING (1 sequence)

SEQ ID NO: 1 - DNA, 215 bp, Vitis vinifera
cacaactttcactatggatgcgccttctcrtcttgtttttctccctttgttctcctctcactatctttctccttttttccatgagcttaattgtcaagaaaactgcagaaatgggggttcctttgcagcccaaaacaactccatcgctgaagaagatgatgaacttcatgctccttgttttggactgaagggagctcctagttcttctcttttcc